(19) United States
(12) Patent Application Publication
(10) Pub. No.: US 2026/0179741 A1
(43) Pub. Date: Jun. 25, 2026

(54) SYSTEM FOR DYSPHAGIA PATIENT, METHOD FOR DYSPHAGIA PATIENT, AND PROGRAM FOR DYSPHAGIA PATIENT

(71) Applicant: SHIMADZU CORPORATION, Kyoto-shi (JP)

(72) Inventor: Takahiro KAWAMURA, Kyoto-shi (JP)

(73) Assignee: SHIMADZU CORPORATION, Kyoto-shi (JP)

(21) Appl. No.: 19/418,425

(22) Filed: Dec. 12, 2025

(30) Foreign Application Priority Data
Dec. 24, 2024 (JP) 2024-227510

(51) Int. Cl. G16H 10/60; G16H 20/00; G16H 30/20

(52) U.S. Cl. CPC G16H 10/60; G16H 20/00; G16H 30/20

(57) ABSTRACT

A system for a dysphagia patient 100 includes: an information acquisition unit 10 that acquires patient identification information 30 capable of identifying a patient and bed angle information 31 of the patient's bed 6 based on the patient's swallowing function; a storage unit 11 in which the patient identification information 30, location information 32 indicating one of an address of the bed 6, an address of a display device 3 installed in a location visible from the bed 6, and the patient's whereabouts, and the angle information 31 are stored in association with each other; and a control unit 12. The control unit 12 uses the patient identification information 30 to acquire the location information 32 and the angle information 31 associated with the patient identification information 30 from the storage unit 11, and uses the location information 32 to transmit the angle information 31 to at least one of the bed 6, the display device 3, and an information terminal 8 held by a caregiver for the patient's meal.

### TECHNICAL FIELD

The present invention relates to a system for a dysphagia patient, a method for a dysphagia patient, and a program for a dysphagia patient, and particularly to a system for a dysphagia patient, a method for a dysphagia patient, and a program for a dysphagia patient for a patient with an impaired swallowing function.

### BACKGROUND ART

Conventionally, an X-ray image diagnostic apparatus for inspecting the swallowing function of a patient with an impaired swallowing function is known (for example, see Patent Literature 1).

The X-ray image diagnostic apparatus described in Patent Literature 1 includes an imaging apparatus and an operation apparatus. The operation apparatus includes an X-ray source, an X-ray collimator, and an X-ray image receptor. The operation apparatus also includes an image processing device. In the configuration disclosed in Patent Literature 1, X-rays irradiated from the X-ray source are detected by the X-ray image receptor, and an electrical signal corresponding to the intensity of the detected X-rays is transmitted to the image processing device. The image processing device generates an X-ray image based on the transmitted electrical signal. Further, the image processing device uses the generated X-ray image to detect the movement of the hyoid bone of a subject and determines the swallowing function of the subject.

### Citation List

### Patent Literature

[Patent Literature 1] Japanese Unexamined Patent Application Publication No. 2018-15263

### SUMMARY OF INVENTION

### Technical Problem

Here, although not disclosed in Patent Literature 1, a doctor or a speech-language pathologist determines the angle of the bed for when a patient (subject) has a meal, according to the swallowing function of the subject, and records it in a memo or the like. Then, a nurse or the like adjusts the bed to an angle corresponding to the patient's swallowing function based on the memo or the like written by the doctor or speech-language pathologist when the patient has a meal. However, when transmitting information on the bed angle by a memo or the like written by a doctor or speech-language pathologist, there is a problem in that human error is likely to occur because the bed angle information is transmitted via a person.

The present invention has been made to solve the above-described problems, and an object of the present invention is to provide a system for a dysphagia patient, a method for a dysphagia patient, and a program for a dysphagia patient that can suppress the occurrence of human error related to the transmission of bed angle information when adjusting the bed angle of a patient with an impaired swallowing function.

### Solution to Problem

To achieve the above object, a system for a dysphagia patient according to a first aspect of the present invention includes an information acquisition unit that acquires patient identification information capable of identifying a patient from an X-ray imaging apparatus and bed angle information of the patient's bed set based on the patient's swallowing function, a storage unit in which the patient identification information, location information indicating one of an address of the bed, an address of a display device installed in a location visible from the bed, and the patient's whereabouts, and the angle information are stored in association with each other, and a control unit. The control unit uses the patient identification information to acquire the location information and the angle information associated with the patient identification information from the storage unit, and uses the acquired location information to transmit the angle information to at least one of the bed, the display device, and an information terminal held by a caregiver for the patient's meal.

A method for a dysphagia patient according to a second aspect of the present invention includes the steps of: acquiring patient identification information capable of identifying a patient from an X-ray imaging apparatus and bed angle information of the patient's bed set based on the patient's swallowing function; storing the patient identification information, location information indicating one of an address of the bed, an address of a display device installed in a location visible from the bed, and the patient's whereabouts, and the angle information in association with each other; using the patient identification information to acquire the location information and the angle information associated with the patient identification information; and using the acquired location information to transmit the angle information to at least one of the bed, the display device, and an information terminal held by a caregiver for the patient's meal.

A program for a dysphagia patient according to a third aspect of the present invention causes a computer to execute the controls of: acquiring patient identification information capable of identifying a patient from an X-ray imaging apparatus and bed angle information of the patient's bed set based on the patient's swallowing function; storing the patient identification information, location information indicating one of an address of the bed, an address of a display device installed in a location visible from the bed, and the patient's whereabouts, and the angle information in association with each other; using the patient identification information to acquire the location information and the angle information associated with the patient identification information; and using the acquired location information to transmit the angle information to at least one of the bed, the display device, and an information terminal held by a caregiver for the patient's meal.

### Advantageous Effects of Invention

In the system for a dysphagia patient according to the first aspect, the method for a dysphagia patient according to the second aspect, and the program for a dysphagia patient according to the third aspect, as described above, the bed angle information of the patient set based on the patient's swallowing function is transmitted to at least one of the patient's bed, the display device installed in a location visible from the bed, and the information terminal held by the caregiver for the patient's meal, using the location information associated with the patient identification information. This makes it possible to transmit the bed angle information from a doctor or a speech-language pathologist to a nurse or the like without involving a person using a memo or the like. As a result, it is possible to suppress the occurrence of human error related to the transmission of bed angle information when adjusting the bed angle of a patient with an impaired swallowing function.

### BRIEF DESCRIPTION OF DRAWINGS

FIG. 1 is a block diagram showing a configuration of a system for a dysphagia patient according to a first embodiment.

FIG. 2 is a block diagram showing a configuration in which a control unit according to the first embodiment transmits angle information and food form information to a display device.

FIG. 3 is a diagram for explaining a configuration in which a display device according to the first embodiment displays bed angle information and food form information.

FIG. 4 is a diagram for explaining a configuration in which a display device according to the first embodiment notifies that a bed angle is incorrect.

FIG. 5 is a flowchart for explaining a process in which a control unit according to the first embodiment stores bed angle information and food form information in association with patient identification information.

FIG. 6 is a flowchart for explaining a process in which a control unit according to the first embodiment transmits bed angle information and food form information to a display device.

FIG. 7 is a flowchart for explaining a process in which a control unit according to the first embodiment notifies that a bed angle is incorrect.

FIG. 8 is a block diagram showing a configuration of a system for a dysphagia patient according to a second embodiment.

FIG. 9 is a block diagram showing a configuration in which a control unit according to the second embodiment transmits angle information to an angle adjustment mechanism and transmits food form information to a display device.

FIG. 10 is a flowchart for explaining a process in which a control unit according to the second embodiment transmits bed angle information to an angle adjustment mechanism and transmits food form information to a display device.

FIG. 11 is a block diagram showing a configuration of a system for a dysphagia patient according to a third embodiment.

FIG. 12 is a block diagram showing a configuration in which a control unit according to the third embodiment transmits angle information and food form information to an information terminal.

FIG. 13 is a block diagram for explaining a configuration in which a control unit according to the third embodiment stores contact information of a caregiver or a patient in association with patient identification information.

FIG. 14 is a flowchart for explaining a process in which a control unit according to the third embodiment transmits bed angle information and food form information to an information terminal.

FIG. 15 is a flowchart for explaining a process in which a control unit according to the third embodiment stores contact information of a caregiver or a patient in association with patient identification information.

FIG. 16 is a diagram for explaining a configuration in which a display device according to a modification displays bed angle information, food form information, and an illustration showing the angle of a bed.

### DESCRIPTION OF EMBODIMENTS

Hereinafter, an embodiment embodying the present invention will be described based on the drawings.

### First Embodiment

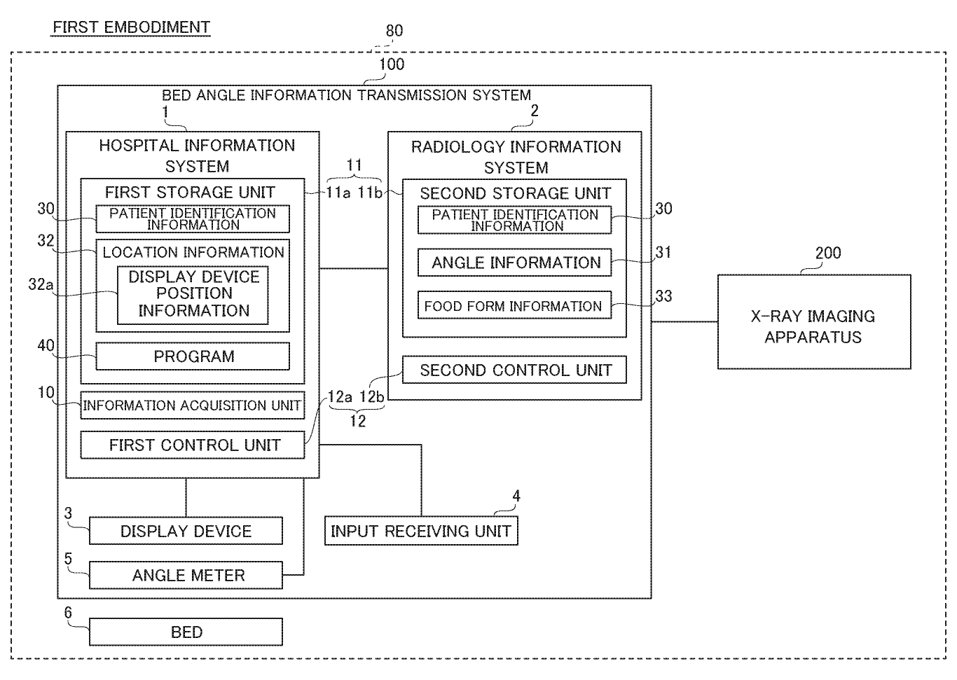

With reference to FIG. 1 to FIG. 7, a configuration of a system for a dysphagia patient 100 according to a first embodiment will be described.

### Overall Configuration of System for Dysphagia Patient

As shown in FIG. 1, the system for a dysphagia patient 100 includes a hospital information system 1, a radiology information system 2, a display device 3, an input receiving unit 4, an angle meter 5, an information acquisition unit 10, a storage unit 11, and a control unit 12.

The system for a dysphagia patient 100 is used to adjust the angle of a patient's bed 6 by transmitting bed angle information 31 of the patient's bed 6, which is set based on the patient's swallowing function, to the display device 3 when a patient with an impaired swallowing function has a meal. The patient's swallowing function is diagnosed by a doctor, a speech-language pathologist, or the like by examining the patient with an X-ray imaging apparatus 200.

As shown in FIG. 1, the system for a dysphagia patient 100, the X-ray imaging apparatus 200, and the bed 6 are arranged in the same facility 80. The facility 80 is, for example, a hospital. The system for a dysphagia patient 100 is a system used to support a caregiver when assisting a patient with an impaired swallowing function with a meal.

The information acquisition unit 10 is configured to acquire patient identification information 30 capable of identifying a patient from the X-ray imaging apparatus 200 and bed angle information 31 of the patient's bed 6 based on the patient's swallowing function. The information acquisition unit 10 is, for example, an input/output interface.

The patient identification information 30 is an ID for identifying a patient in the hospital information system 1. That is, the patient identification information 30 is a user ID in the hospital information system 1. The patient identification information 30 is created when the patient visits the facility 80 such as a hospital for the first time.

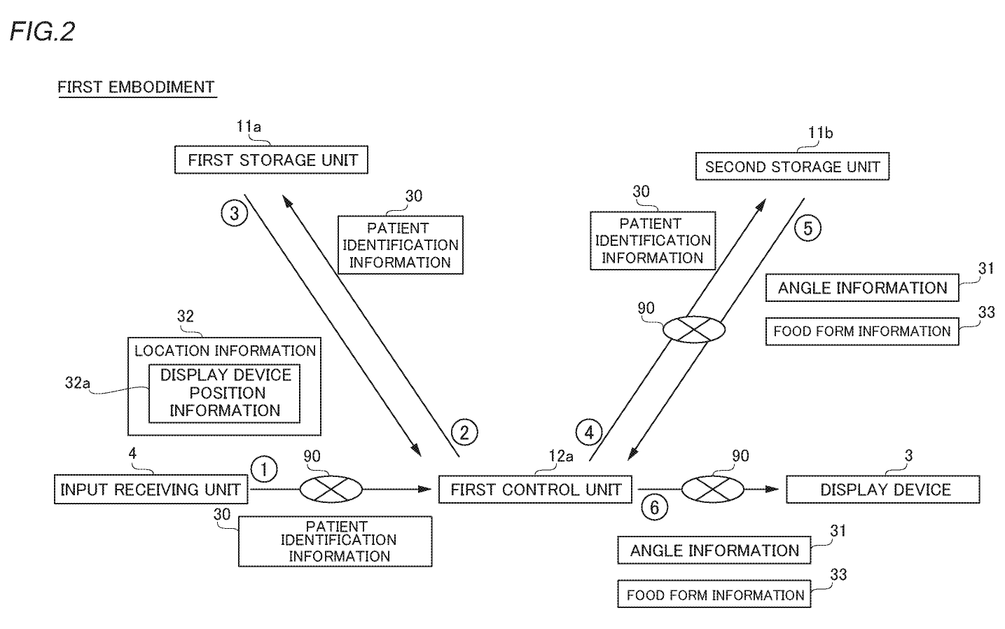

In the storage unit 11, the patient identification information 30, location information 32 indicating an address of the display device 3 installed in a location visible from the bed 6, and the angle information 31 are stored in association with each other. In the first embodiment, the location information 32 includes position information 32a of the display device 3. The position information 32a of the display device 3 includes, for example, information on the room (patient room) where the display device 3 is arranged, an IP address for transmitting the angle information 31 or the like to the display device 3 via a network 90 (see FIG. 2), and the like.

Further, in the first embodiment, in the storage unit 11, food form information 33 and a program 40 are stored together with the patient identification information 30, the angle information 31, and the location information 32. In the first embodiment, in the storage unit 11, food form information 33 corresponding to the patient's swallowing function is stored in association with the patient identification information 30. The food form information 33 is, for example, information indicating whether the meal provided to the patient is chopped food, liquid food, jelly-like food, or regular food (general diet).

In the first embodiment, the storage unit 11 includes a first storage unit 11a included in the hospital information system 1 and a second storage unit 11b included in the radiology information system 2. Further, in the first embodiment, the control unit 12 includes a first control unit 12a included in the hospital information system 1 and a second control unit 12b included in the radiology information system 2.

The program 40 is a program for a dysphagia patient. In the first embodiment, by causing the control unit 12 to execute the program 40, a method for a dysphagia patient can be implemented.

The control unit 12 uses the patient identification information 30 to acquire the location information 32 and the angle information 31 associated with the patient identification information 30 from the storage unit 11. Then, the control unit 12 uses the acquired location information 32 to transmit the angle information 31 to the display device 3.

Further, the control unit 12 uses the patient identification information 30 to acquire the food form information 33 associated with the patient identification information 30 from the storage unit 11, together with the location information 32 and the angle information 31. Then, the control unit 12 transmits the acquired food form information 33 to the display device 3, together with the angle information 31.

The control unit 12 includes, for example, a processor such as a CPU (Central Processing Unit) and a memory such as a ROM (Read Only Memory) and a RAM (Random Access Memory). The control unit 12 includes a first control unit 12a provided in the hospital information system 1 and a second control unit 12b provided in the radiology information system 2. Details of the configuration in which the control unit 12 transmits the angle information 31 and the food form information 33 will be described later.

The hospital information system 1 includes the information acquisition unit 10, the first storage unit 11a, and the first control unit 12a. The hospital information system 1 includes a reception system, an electronic medical record system, an admission/discharge management system, an accounting system, and the like.

In the first embodiment, the patient identification information 30 and the location information 32 are stored in association with each other in the first storage unit 11a.

The radiology information system 2 includes the second storage unit 11b and the second control unit 12b. The radiology information system 2 is a system that manages reservations for examinations by the X-ray imaging apparatus 200 and the like, examination results, and the like.

The second control unit 12b controls each unit of the radiology information system 2. The second control unit 12b includes a processor such as a CPU and a memory such as a ROM and a RAM.

In the second storage unit 11b, the patient identification information 30 and the angle information 31 are stored in association with each other.

The display device 3 is provided in the vicinity of the bed 6. The display device 3 is configured to display the angle information 31. Further, in the first embodiment, the display device 3 is configured to display the food form information 33. The display device 3 includes, for example, a liquid crystal monitor or the like.

The input receiving unit 4 is configured to receive an operation input from an operator. The input receiving unit 4 includes, for example, an input device such as a keyboard and a mouse, a touch panel integrally formed with the display device 3, and the like.

The angle meter 5 is configured to acquire the angle of the bed 6. The angle meter 5 is provided on the bed 6 together with the display device 3, and is configured to move together with the display device 3 when the angle of the bed 6 is changed. The angle meter 5 includes, for example, an angle measurement sensor such as a gyro sensor.

As shown in FIG. 1, the bed 6 or the display device 3 is provided in the same facility 80 as the facility where the storage unit 11 is provided. In the example shown in FIG. 1, the display device 3, the bed 6, and the storage unit 11 are provided in the same facility 80.

The X-ray imaging apparatus 200 includes an unillustrated X-ray source, an X-ray detector, an image processing unit, and an imaging apparatus control unit. X-rays irradiated from the X-ray source are detected by the X-ray detector and converted into an electrical signal. The electrical signal is transmitted to the image processing unit. The image processing unit generates an X-ray image based on the transmitted electrical signal. The imaging apparatus control unit controls each unit of the X-ray imaging apparatus 200.

The hospital information system 1, the radiology information system 2, the display device 3, the input receiving unit 4, the angle meter 5, and the X-ray imaging apparatus 200 are communicably connected by a network 90 in the facility 80. The network 90 is, for example, a LAN (Local Area Network).

### Transmission of Angle Information and Food Form Information

Next, with reference to FIG. 2, a configuration in which the control unit 12 (first control unit 12a) transmits the angle information 31 and the food form information 33 to the display device 3 will be described. In FIG. 2, the numbers enclosed in circles indicate the order of processing.

First, the first control unit 12a acquires the patient identification information 30 input via the input receiving unit 4 (first process). Next, the first control unit 12a uses the patient identification information 30 input via the input receiving unit 4 to acquire the location information 32 and the angle information 31 associated with the patient identification information 30 from the storage unit 11. Specifically, the first control unit 12a uses the patient identification information 30 to acquire the location information 32 (position information 32a of the display device 3) associated with the patient identification information 30 from the first storage unit 11a (second and third processes). Further, the first control unit 12a uses the patient identification information 30 to acquire the angle information 31 associated with the patient identification information 30 from the second storage unit 11b (fourth and fifth processes). In the first embodiment, the first control unit 12a also acquires the food form information 33 together with the angle information 31. That is, when transmitting the angle information 31 to the display device 3, the first control unit 12a acquires the position information 32a of the display device 3, the angle information 31, and the food form information 33 associated with the patient identification information 30.

Thereafter, the first control unit 12a uses the acquired position information 32a of the display device 3 to transmit the angle information 31 to the display device 3 (sixth process). In the first embodiment, the first control unit 12a is configured to transmit the angle information 31 to the display device 3 provided in the same facility 80 as the facility where the storage unit 11 is provided. At this time, the first control unit 12a transmits the acquired food form information 33 to the display device 3 together with the angle information 31.

The display device 3 displays the received angle information 31 and food form information 33.

### Display of Angle Information and Food Form Information

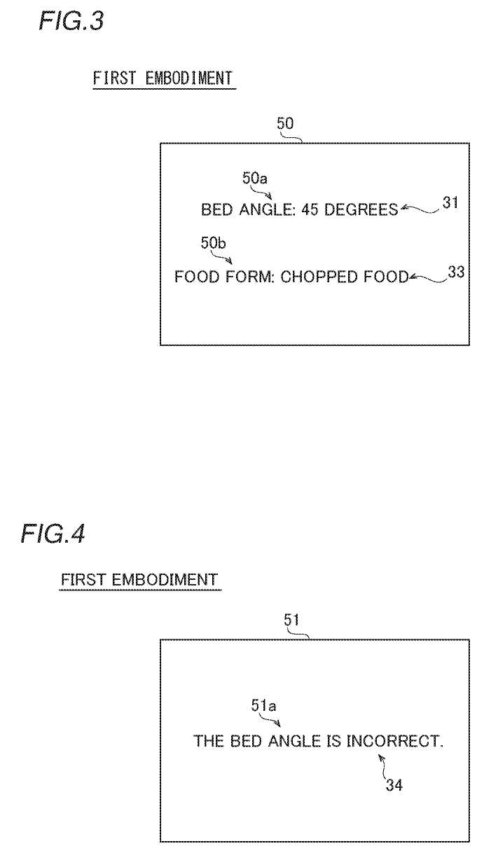

As shown in FIG. 3, the display device 3 is configured to display the transmitted angle information 31. Further, in the first embodiment, the display device 3 is configured to display the food form information 33 together with the angle information 31.

A screen 50 shown in FIG. 3 is an example of a screen when the display device 3 displays the angle information 31 and the food form information 33. The screen 50 includes a display field 50a for the angle information 31 and a display field 50b for the food form information 33.

A caregiver such as a nurse adjusts the angle of the patient's bed 6 while checking the angle information 31 of the bed 6 displayed on the screen 50. Then, after the adjustment of the angle of the bed 6 is completed, the caregiver assists the patient with a meal. At that time, by checking the food form information 33 displayed on the screen 50 and the meal actually served to the patient, the caregiver can confirm whether a meal of an incorrect food form has been served to the patient.

### Notification that Bed Angle is Incorrect

Further, in the first embodiment, the first control unit 12a is configured to, when the angle of the bed 6 acquired by the angle meter 5 is different from the angle information 31, notify that the angle of the bed 6 is set to an incorrect angle.

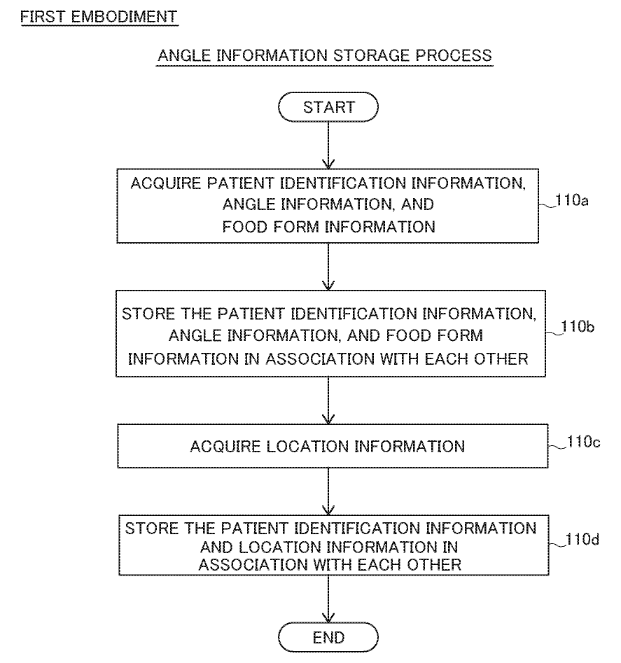

Specifically, as shown in FIG. 4, the first control unit 12a notifies that the angle of the bed 6 is set to an incorrect angle by displaying a screen 51 indicating that the angle of the bed 6 is incorrect on the display device 3.

A screen 51 is an example of a screen when the display device 3 notifies that the angle of the bed 6 is incorrect. The screen 51 includes a display field 51a for displaying a message 34 indicating that the angle of the bed 6 is incorrect.

If the angle of the bed 6 is incorrect, the caregiver can grasp that fact by checking the message 34 displayed on the screen 51. Therefore, the angle of the bed 6 can be re-adjusted to the correct angle.

### Update of Angle Information

In the first embodiment, the control unit 12 is configured to, when repeating the examination by the X-ray imaging apparatus 200, acquire the angle information 31 stored in the storage unit 11, and if there is a change in the angle information 31 as a result of the examination of the patient's swallowing function, update the angle information 31. Specifically, when performing an examination of the patient's swallowing function, the second control unit 12b uses the patient identification information 30 to acquire the angle information 31 stored in the second storage unit 11b. Then, if the bed angle information 31 of the patient's bed 6 is changed as a result of the examination by the X-ray imaging apparatus 200, the second control unit 12b overwrites the angle information 31 stored in the second storage unit 11b with new angle information 31.

### Angle Information Storage Process

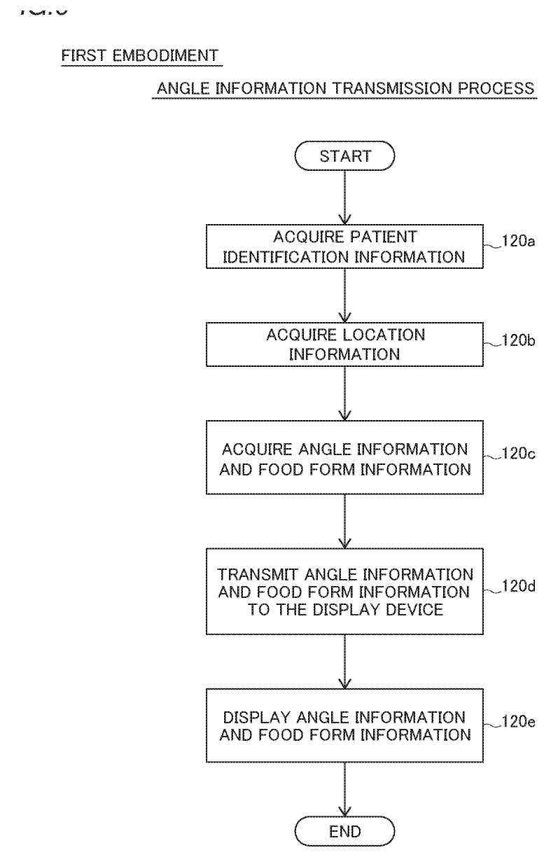

Next, with reference to FIG. 5, a process in which the first control unit 12a (see FIG. 1) and the second control unit 12b (see FIG. 1) store the angle information 31 (see FIG. 1) in the storage unit 11 (see FIG. 1) in association with the patient identification information 30 (see FIG. 1) will be described.

In step 110a, the second control unit 12b acquires the patient identification information 30 capable of identifying a patient from the X-ray imaging apparatus 200 and the bed angle information 31 of the patient's bed 6 based on the patient's swallowing function. In the first embodiment, in the process of step 110a, the second control unit 12b also acquires the food form information 33 together with the patient identification information 30 and the angle information 31.

Next, in step 110b, the second control unit 12b stores the patient identification information 30, the angle information 31, and the food form information 33 in the storage unit 11 in association with each other. In the first embodiment, in the process of step 110b, the second control unit 12b stores the patient identification information 30, the angle information 31, and the food form information 33 in the second storage unit 11b (see FIG. 1) of the radiology information system 2 in association with each other.

Next, in step 110c, the first control unit 12a acquires the location information 32. Specifically, the first control unit 12a acquires the location information 32 corresponding to the patient identification information 30 by an operator's operation input or the like. In the first embodiment, the first control unit 12a acquires the position information 32a of the display device 3 as the location information 32.

Next, in step 110d, the first control unit 12a stores the patient identification information 30 and the location information 32 in association with each other. Specifically, the first control unit 12a stores the patient identification information 30 and the location information 32 in the first storage unit 11a (see FIG. 1) of the hospital information system 1 in association with each other. Thereafter, the process ends.

In the first embodiment, by performing the processes of steps 110a to 110d, the control unit 12 stores the location information 32 indicating the address of the display device 3 and the angle information 31 in the storage unit 11 in association with each other.

### Angle Information Transmission Process

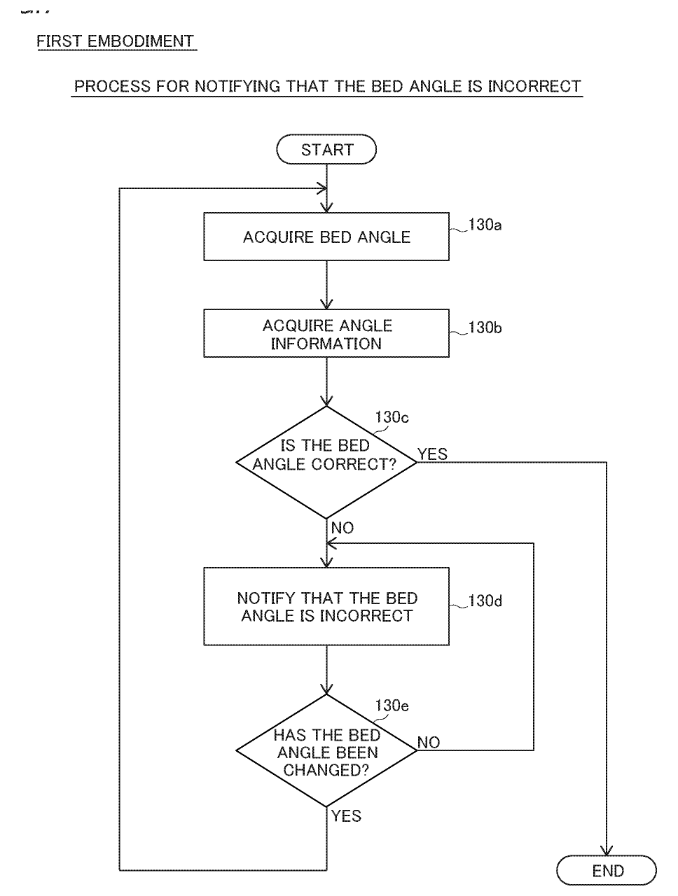

Next, with reference to FIG. 6, a process in which the first control unit 12a (see FIG. 1) and the second control unit 12b (see FIG. 1) transmit the angle information 31 (see FIG. 1) to the display device 3 (see FIG. 1) will be described.

In step 120a, the first control unit 12a acquires the patient identification information 30 (see FIG. 1). Specifically, the first control unit 12a acquires the patient identification information 30 based on an operation input from an operator (caregiver) to the input receiving unit 4 (see FIG. 1). For example, when the caregiver performs an operation to provide meal assistance to the patient, the patient identification information 30 is transmitted to the first control unit 12a via the input receiving unit 4. The first control unit 12a acquires the transmitted patient identification information 30. The process of step 120a is the first process in FIG. 2.

Next, in step 120b, the first control unit 12a uses the patient identification information 30 to acquire the location information 32 (see FIG. 1). Specifically, the first control unit 12a acquires the location information 32 associated with the patient identification information 30 from the first storage unit 11a (see FIG. 1). In the first embodiment, the first control unit 12a acquires the position information 32a of the display device 3 (see FIG. 1) associated with the patient identification information 30 from the first storage unit 11a. The process of step 120b is the second and third processes in FIG. 2.

Next, in step 120c, the first control unit 12a uses the patient identification information 30 to acquire the angle information 31. Specifically, the first control unit 12a transmits the patient identification information 30 to the second control unit 12b. The second control unit 12b acquires the angle information 31 associated with the received patient identification information 30 from the second storage unit 11b (see FIG. 1). Then, the second control unit 12b transmits the acquired angle information 31 to the first control unit 12a. Thereby, the first control unit 12a acquires the angle information 31.

Further, in the first embodiment, in step 120c, the first control unit 12a uses the patient identification information 30 to acquire the food form information 33 (see FIG. 1). Specifically, the first control unit 12a transmits the patient identification information 30 to the second control unit 12b. The second control unit 12b acquires the food form information 33 associated with the received patient identification information 30 from the second storage unit 11b. Then, the second control unit 12b transmits the acquired food form information 33 to the first control unit 12a. Thereby, the first control unit 12a acquires the food form information 33. The process of step 120c is the fourth and fifth processes in FIG. 2.

In the first embodiment, in step 120b and step 120c, the first control unit 12a uses the patient identification information 30 to acquire the location information 32 and the angle information 31 associated with the patient identification information 30.

Next, in step 120d, the first control unit 12a uses the acquired location information 32 to transmit the angle information 31 to the display device 3. In the first embodiment, the first control unit 12a uses the position information 32a of the display device 3 to transmit the angle information 31 and the food form information 33 to the display device 3. The process of step 120e is the sixth process in FIG. 2.

Next, in step 120e, the display device 3 displays the angle information 31 and the food form information 33. Thereafter, the process ends.

The process of step 120b and the process of step 120c may be executed in any order.

(Process for Notifying that Bed Angle is Incorrect)

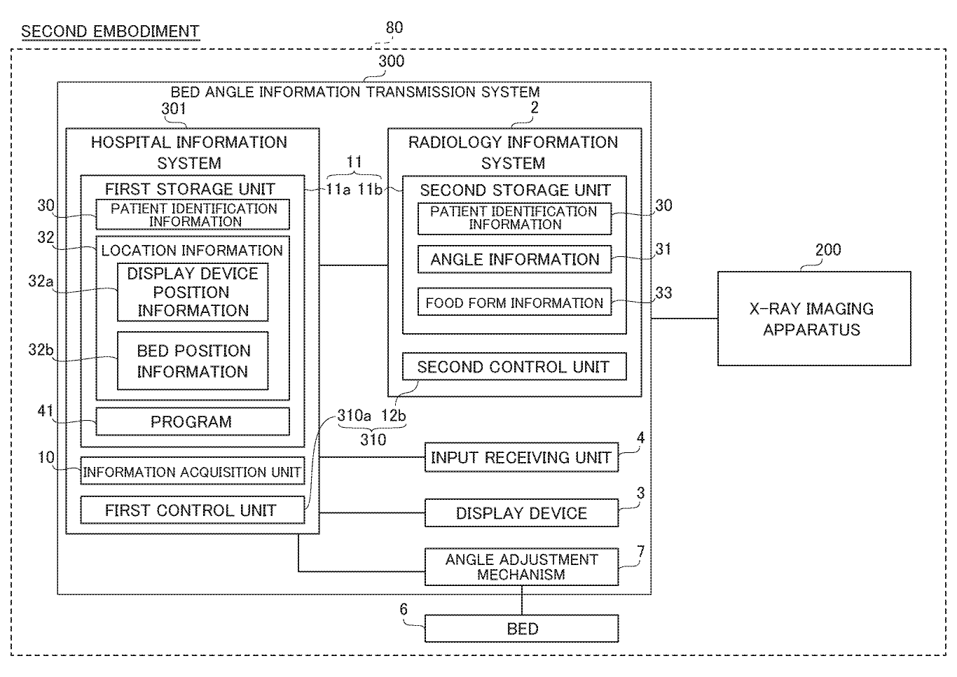

Next, with reference to FIG. 7, a process in which the first control unit 12a (see FIG. 1) notifies that the angle of the bed 6 (see FIG. 1) is incorrect will be described. The process shown in FIG. 7 is executed after the angle of the bed 6 has been adjusted by the caregiver.

In step 130a, the first control unit 12a acquires the angle of the bed 6. Specifically, the first control unit 12a acquires information on the current angle of the bed 6 from the angle meter 5 (see FIG. 1).

Next, in step 130b, the first control unit 12a acquires the angle information 31 (see FIG. 1). Specifically, the first control unit 12a uses the patient identification information 30 (see FIG. 1) to acquire the angle information 31 from the second storage unit 11b (see FIG. 1).

Next, in step 130c, the first control unit 12a determines whether the angle of the bed 6 is correct. Specifically, the first control unit 12a determines whether the angle of the bed 6 is correct based on whether the information on the current angle of the bed 6 acquired in step 130a matches the angle information 31 acquired in step 130b. If the angle of the bed 6 is correct, the process ends. If the angle of the bed 6 is not correct, the process proceeds to step 130d.

When the process proceeds from step 130c to step 130d, in step 130d, the first control unit 12a notifies that the angle of the bed 6 is incorrect. Specifically, the first control unit 12a notifies that the angle of the bed 6 is incorrect by displaying the message 34 indicating that the angle of the bed 6 is incorrect on the screen 51 shown in FIG. 4.

Next, in step 130e, the first control unit 12a determines whether the angle of the bed 6 has been changed. If the angle of the bed 6 has been changed, the process proceeds to step 130a. If the angle of the bed 6 has not been changed, the process proceeds to step 130d.

### Second Embodiment

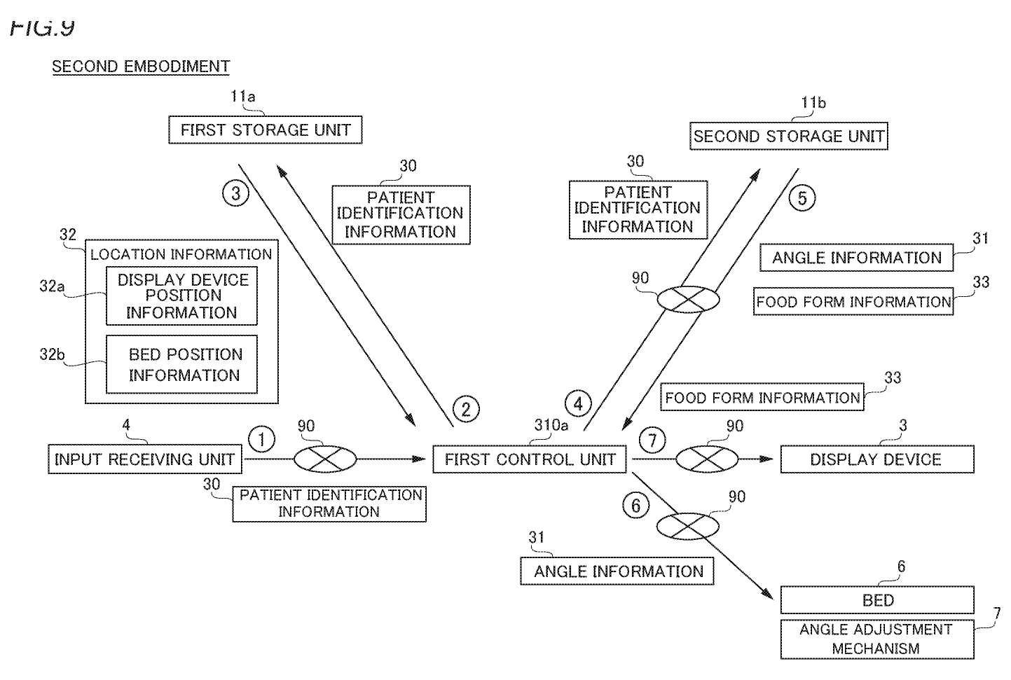

Next, a second embodiment will be described with reference to FIG. 8 to FIG. 10. In a system for a dysphagia patient 300 according to the second embodiment, a configuration in which the angle of the bed 6 is automatically adjusted by an angle adjustment mechanism 7 will be described. Note that components similar to those in the first embodiment described above are denoted by the same reference signs, and detailed description thereof is omitted.

### Overall Configuration of System for Dysphagia Patient

As shown in FIG. 8, the system for a dysphagia patient 300 according to the second embodiment includes a hospital information system 301, the radiology information system 2, the display device 3, the input receiving unit 4, an angle adjustment mechanism 7, the storage unit 11, and a control unit 310. The hospital information system 301, the radiology information system 2, the display device 3, the input receiving unit 4, the bed 6, the angle adjustment mechanism 7, and the X-ray imaging apparatus 200 are provided in the same facility 80.

In the second embodiment, the control unit 310 includes a first control unit 310a and the second control unit 12b.

The hospital information system 301 includes the information acquisition unit 10, the first storage unit 11a, and a first control unit 310a. The radiology information system 2 includes the second storage unit 11b and the second control unit 12b.

The first control unit 310a uses the patient identification information 30 to acquire the location information 32 and the angle information 31 associated with the patient identification information 30 from the storage unit 11. Then, the first control unit 310a uses the acquired location information 32 to transmit the angle information 31 to the bed 6. When transmitting the angle information 31 to the bed 6, the first control unit 310a is configured to transmit the angle information 31 to the angle adjustment mechanism 7.

The angle adjustment mechanism 7 uses the transmitted angle information 31 to adjust the angle of the bed 6. Specifically, the angle adjustment mechanism 7 is arranged at a movable part of the bed 6, and adjusts the angle of the bed 6 by moving the movable part of the bed 6 using the angle information 31. The angle adjustment mechanism 7 includes, for example, a drive unit and a transmission member.

Further, in the second embodiment, in the storage unit 11, the patient identification information 30, location information 32 indicating an address of the bed 6, and the angle information 31 are stored in association with each other. In the second embodiment, the location information 32 includes position information 32b of the bed 6. The position information 32b of the bed 6 includes, for example, information on the room (patient room) where the bed 6 is arranged, an IP address for transmitting the angle information 31 or the like to the bed 6 (angle adjustment mechanism 7) via the network 90 (see FIG. 2), and the like. In the second embodiment, the position information 32b of the bed 6 includes the IP address of the angle adjustment mechanism 7.

Further, in the second embodiment, the patient identification information 30, the location information 32, and a program 41 are stored in the first storage unit 11a. The patient identification information 30, the angle information 31, and the food form information 33 are stored in the second storage unit 11b.

The program 41 is a program for a dysphagia patient. In the second embodiment, by causing the control unit 12 to execute the program 41, a method for a dysphagia patient can be implemented.

In the second embodiment, the hospital information system 1, the radiology information system 2, the display device 3, the input receiving unit 4, the angle adjustment mechanism 7, and the X-ray imaging apparatus 200 are communicably connected by the network 90 in the facility 80.

### Storage of Location Information

In the second embodiment, the first control unit 310a and the second control unit 12b store the patient identification information 30, the location information 32 indicating the address of the bed 6, and the angle information 31 in association with each other. Specifically, similarly to the first control unit 12a and the second control unit 12b according to the first embodiment described above, the first control unit 310a and the second control unit 12b acquire the patient identification information 30, the angle information 31, and the location information 32 (position information 32b of the bed 6), and store the acquired patient identification information 30, angle information 31, and position information 32b of the bed 6 in the storage unit 11 in association with each other.

### Transmission of Angle Information and Food Form Information

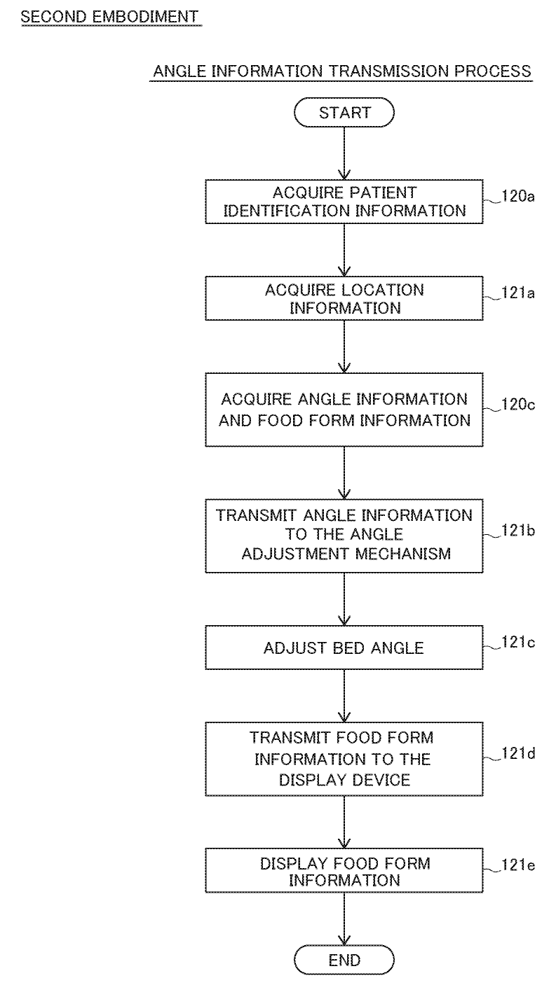

Next, with reference to FIG. 9, a configuration in which the control unit 310 (first control unit 310a) transmits the angle information 31 to the bed 6 will be described. In FIG. 9, the numbers enclosed in circles indicate the order of processing.

First, the control unit 310 acquires the patient identification information 30 input via the input receiving unit 4 (first process). Next, the first control unit 310a uses the patient identification information 30 input via the input receiving unit 4 to acquire the location information 32 and the angle information 31 associated with the patient identification information 30 from the storage unit 11. Specifically, the first control unit 310a acquires the location information 32 (position information 32b of the bed 6) associated with the patient identification information 30 from the first storage unit 11a (second and third processes). In the second embodiment, the first control unit 310a acquires the position information 32a of the display device 3 together with the position information 32b of the bed 6.

Further, the first control unit 310a uses the patient identification information 30 to acquire the location information 32 and the angle information 31 associated with the patient identification information 30 from the second storage unit 11b (fourth and fifth processes). In the second embodiment, when acquiring the angle information 31, the control unit 12 also acquires the food form information 33. That is, when transmitting the angle information 31 to the bed 6, the first control unit 310a acquires the position information 32b of the bed 6 and the angle information 31 associated with the patient identification information 30.

Thereafter, the first control unit 310a uses the acquired position information 32b of the bed 6 to transmit the angle information 31 to the bed 6 (sixth process). In the second embodiment, the first control unit 310a is configured to transmit the angle information 31 to the bed 6 provided in the same facility 80 as the facility where the storage unit 11 is provided. In the second embodiment, the first control unit 310a transmits the angle information 31 to the angle adjustment mechanism 7.

The angle adjustment mechanism 7 uses the received angle information 31 to adjust the angle of the bed 6.

Further, the first control unit 310a transmits the food form information 33 to the display device 3 (seventh process). The display device 3 displays the received angle information 31.

### Angle Information Transmission Process

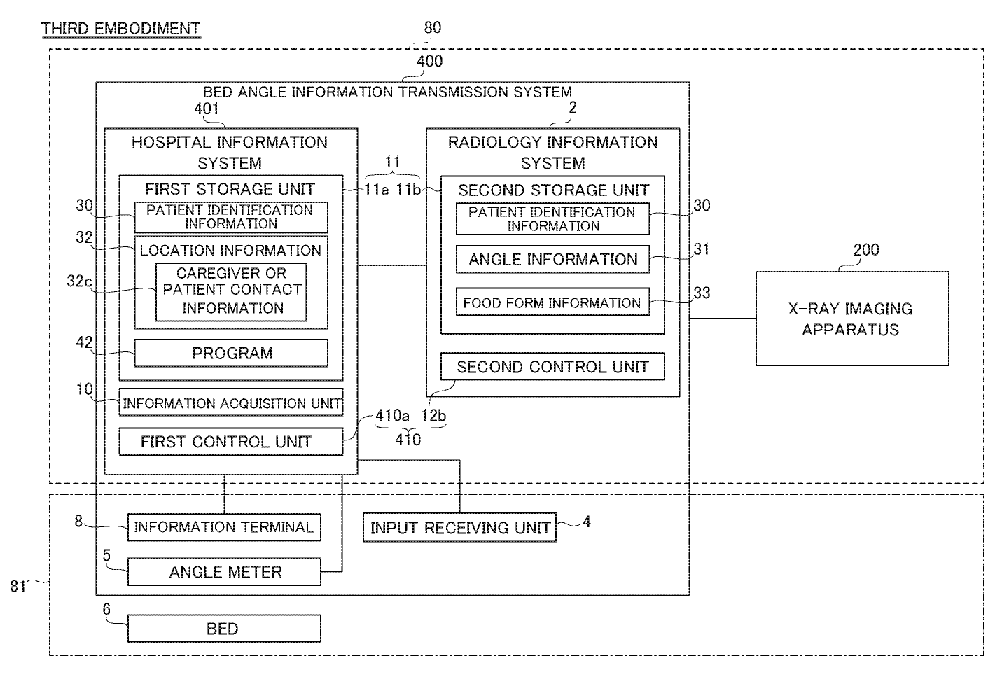

Next, with reference to FIG. 10, a process in which the first control unit 310a (see FIG. 8) and the second control unit 12b (see FIG. 8) transmit the angle information 31 (see FIG. 8) to the bed 6 (see FIG. 8) will be described.

In step 120a, the first control unit 310a acquires the patient identification information 30 (see FIG. 8).

Next, in step 121a, the first control unit 310a uses the patient identification information 30 to acquire the location information 32 (see FIG. 8). In the second embodiment, the first control unit 310a uses the patient identification information 30 to acquire the position information 32a of the display device 3 (see FIG. 8) and the position information 32b of the bed 6 (see FIG. 8). The process of step 121a is the second and third processes in FIG. 9.

Next, in 120c, the first control unit 310a and the second control unit 12b acquire the angle information 31 and the food form information 33 (see FIG. 8).

Next, in step 121b, the first control unit 310a uses the acquired location information 32 to transmit the angle information 31 to the bed 6. Specifically, the first control unit 310a uses the position information 32b of the bed 6 to transmit the angle information 31 to the angle adjustment mechanism 7 provided on the bed 6. Step 121b is the sixth process in FIG. 9.

Next, in step 121c, the angle adjustment mechanism 7 uses the angle information 31 to adjust the angle of the bed 6.

Next, in step 121d, the first control unit 310a uses the position information 32a of the display device 3 to transmit the food form information 33 to the display device 3. Step 121d is the seventh process in FIG. 9.

Next, in step 121e, the display device 3 displays the food form information 33. Thereafter, the process ends.

The process of step 121a and the process of step 120c may be executed in any order. Further, the processes of step 121b and step 121c and the processes of step 121d and step 121e may be executed in any order, or may be processed in parallel.

Other configurations of the second embodiment are the same as those of the first embodiment described above.

### Third Embodiment

Next, a third embodiment will be described with reference to FIG. 11 to FIG. 15. In a system for a dysphagia patient 400 according to the third embodiment, a configuration for transmitting the angle information 31 to an information terminal 8 possessed by a patient's caregiver will be described. Note that components similar to those in the first embodiment described above are denoted by the same reference signs, and detailed description thereof is omitted.

### Overall Configuration of System for Dysphagia Patient

As shown in FIG. 11, the system for a dysphagia patient 400 according to the third embodiment includes a hospital information system 401, the radiology information system 2, the input receiving unit 4, the angle meter 5, an information terminal 8 held by a caregiver for the patient's meal, the storage unit 11, and a control unit 410. The hospital information system 401, the radiology information system 2, and the input receiving unit 4 are provided in the same facility 80. Further, the bed 6 and the information terminal 8 are provided in a facility 81 different from the facility 80. The facility 81 includes, for example, the patient's home, a nursing home, a long-term care facility, and the like.

In the third embodiment, the control unit 410 includes a first control unit 410a and the second control unit 12b.

The hospital information system 401 includes the information acquisition unit 10, a first control unit 410a, and the first storage unit 11a. The radiology information system 2 includes the second storage unit 11b and the second control unit 12b.

The first control unit 410a uses the patient identification information 30 to acquire the location information 32 and the angle information 31 associated with the patient identification information 30 from the storage unit 11. Then, the first control unit 410a uses the acquired location information 32 to transmit the angle information 31 to the information terminal 8.

The information terminal 8 is, for example, a smartphone or a tablet terminal. The information terminal 8 includes a display unit, a control unit, and the like. The information terminal 8 is configured to display the transmitted angle information 31.

Further, in the third embodiment, in the storage unit 11, location information 32 indicating the caregiver's whereabouts and the angle information 31 are stored in association with each other. Specifically, the patient identification information 30, the location information 32, and a program 42 are stored in the first storage unit 11a. The patient identification information 30, the angle information 31, and the food form information 33 are stored in the second storage unit 11b.

In the third embodiment, the location information 32 includes contact information 32c of the caregiver or the patient. The contact information 32c of the caregiver or the patient includes, for example, the caregiver's telephone number, the caregiver's email address, the patient's telephone number, the patient's email address, and the like.

The program 42 is a program for a dysphagia patient. In the third embodiment, by causing the control unit 12 to execute the program 42, a method for a dysphagia patient can be implemented.

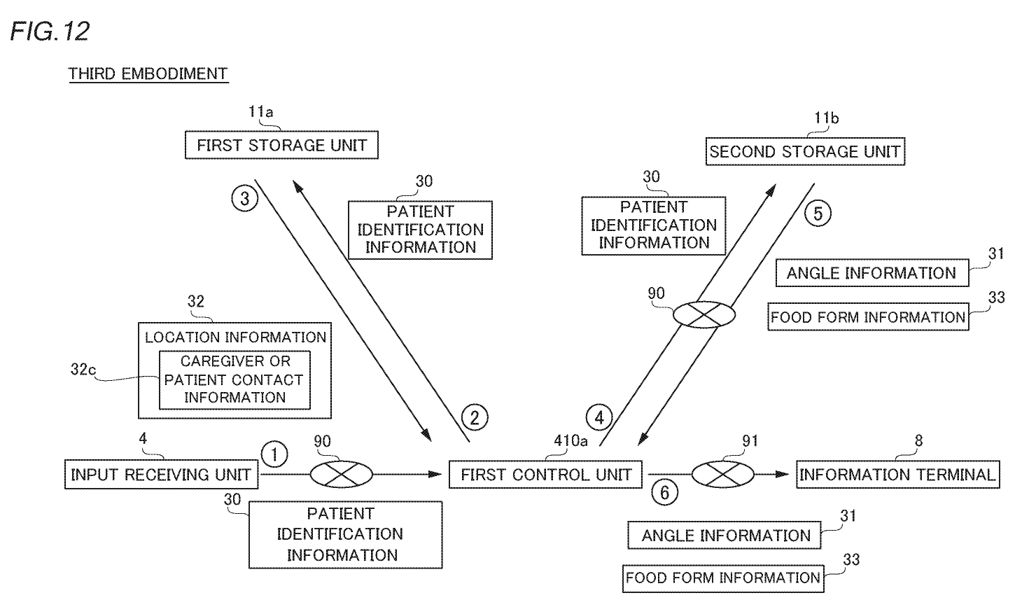

In the third embodiment, the hospital information system 1, the radiology information system 2, and the X-ray imaging apparatus 200 are communicably connected by the network 90 in the facility 80. The input receiving unit 4, the angle meter 5, and the information terminal 8 are communicably connected to the hospital information system 1 via a network 91 (see FIG. 12). The network 91 is, for example, the Internet.

### (Storage of Location Information)

In the third embodiment, the first control unit 410a and the second control unit 12b store the patient identification information 30, the location information 32 indicating the patient's whereabouts, and the angle information 31 in association with each other. Specifically, similarly to the first control unit 12a and the second control unit 12b according to the first embodiment described above, the first control unit 410a and the second control unit 12b acquire the patient identification information 30, the angle information 31, and the location information 32 (contact information 32c of the caregiver or the patient), and store the acquired patient identification information 30, angle information 31, and contact information 32c of the caregiver or the patient in the storage unit 11 in association with each other.

(Transmission of Angle Information and Food Form Information)

Next, with reference to FIG. 12, a configuration in which the control unit 410 (first control unit 410a) transmits the angle information 31 to the information terminal 8 will be described. In FIG. 12, the numbers enclosed in circles indicate the order of processing.

First, the first control unit 410a acquires the patient identification information 30 input via the input receiving unit 4 (first process). Next, the first control unit 410a uses the patient identification information 30 input via the input receiving unit 4 to acquire the location information 32 and the angle information 31 associated with the patient identification information 30 from the storage unit 11. Specifically, the first control unit 410a acquires the location information 32 (contact information 32c of the caregiver or the patient) associated with the patient identification information 30 from the first storage unit 11a (second and third processes). Further, the first control unit 410a uses the patient identification information 30 to acquire the angle information 31 associated with the patient identification information 30 from the second storage unit 11b. Further, the first control unit 410a uses the patient identification information 30 to acquire the food form information 33 associated with the patient identification information 30 from the second storage unit 11b together with the angle information 31 (fourth and fifth processes). That is, when transmitting the angle information 31 to the information terminal 8, the first control unit 410a acquires the contact information 32c and the angle information 31 associated with the patient identification information 30.

Thereafter, the first control unit 410a uses the acquired contact information 32c to transmit the angle information 31 to the information terminal 8 (sixth process). In the third embodiment, the first control unit 410a is configured to transmit the angle information 31 to the information terminal 8 provided in a facility 81 different from the facility 80 where the storage unit 11 is provided. In the third embodiment, the first control unit 410a transmits the acquired food form information 33 to the information terminal 8 together with the angle information 31.

### Registration of Contact Information of Caregiver or Patient

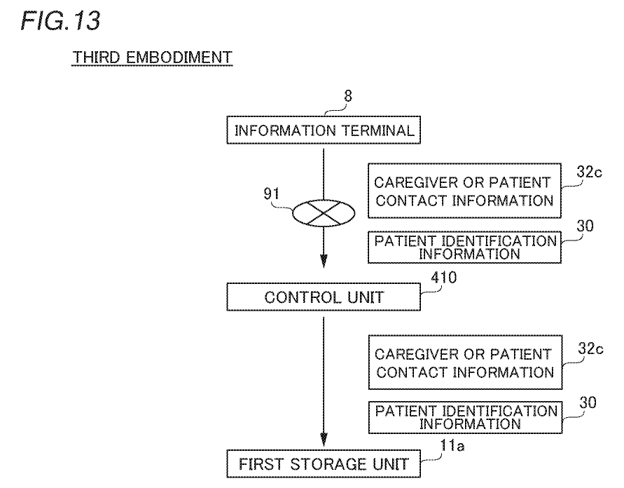

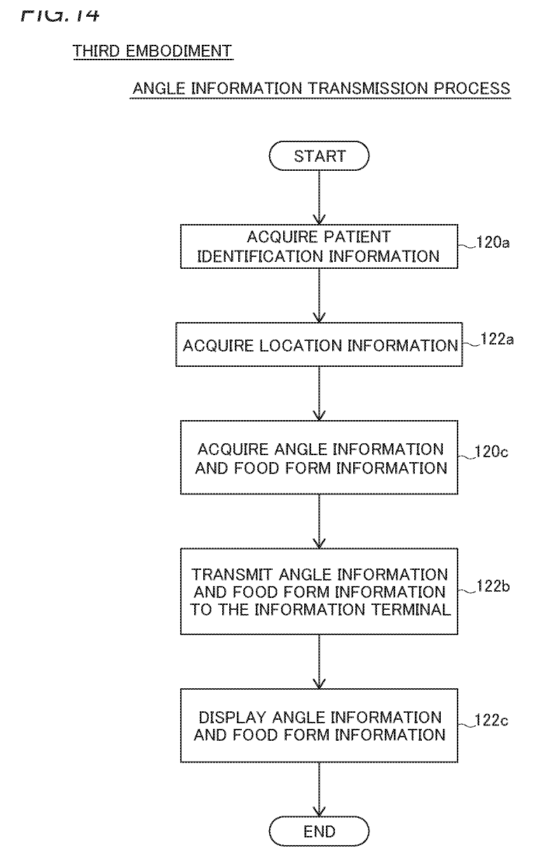

Next, with reference to FIG. 13, a configuration in which the contact information 32c of the caregiver or the patient is stored in the first storage unit 11a will be described.

The contact information 32c of the caregiver or the patient is transmitted from the information terminal 8 to the control unit 410 via the network 91, together with the patient identification information 30. For example, the contact information 32c of the caregiver or the patient is transmitted to the control unit 410 by the caregiver operating an application installed on the information terminal 8. When the caregiver operates the application to register the patient's information, a patient ID in the application associated with the patient identification information 30 is assigned. Therefore, the control unit 410 can identify the patient by the patient identification information 30 and the patient ID in the application.

The control unit 410 stores the patient identification information 30 and the contact information 32c transmitted from the information terminal 8 in the first storage unit 11a in association with each other.

### Angle Information Transmission Process

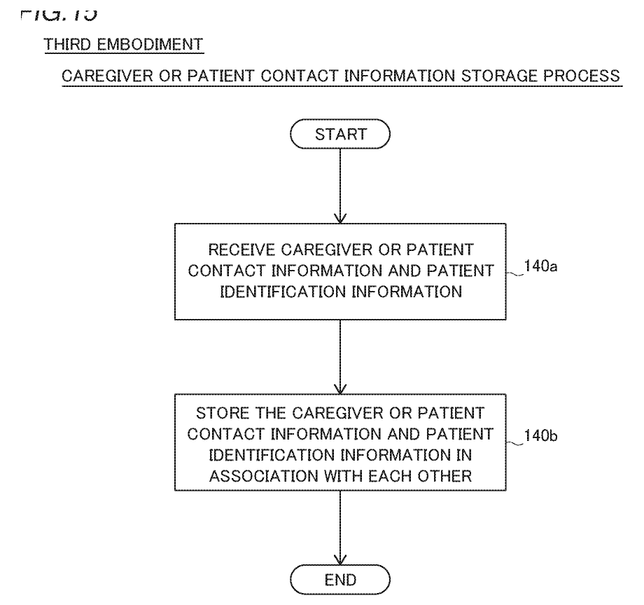

Next, with reference to FIG. 14, a process in which the first control unit 410a (see FIG. 11) and the second control unit 12b (see FIG. 11) transmit the angle information 31 (see FIG. 11) and the food form information 33 (see FIG. 11) to the information terminal 8 (see FIG. 11) will be described.

In step 120a, the first control unit 410a acquires the patient identification information 30.

Next, in step 122a, the first control unit 410a uses the patient identification information 30 to acquire the location information 32. In the third embodiment, the first control unit 410a uses the patient identification information 30 to acquire the contact information 32c of the caregiver or the patient. The process of step 122a is the second and third processes in FIG. 12.

Next, in 120c, the first control unit 410a and the second control unit 12b acquire the angle information 31 and the food form information 33.

Next, in step 122b, the first control unit 410a uses the acquired location information 32 to transmit the angle information 31 to the information terminal 8 held by the caregiver. Specifically, the first control unit 410a uses the contact information 32c to transmit the angle information 31 to the information terminal 8. In the process of step 122b, the first control unit 410a uses the contact information 32c to transmit the food form information 33 to the information terminal 8 together with the angle information 31. The process of step 122b is the sixth process in FIG. 12.

Next, in step 122c, the information terminal 8 displays the angle information 31 and the food form information 33. Thereafter, the process ends.

### Contact Information Storage Process of Caregiver or Patient

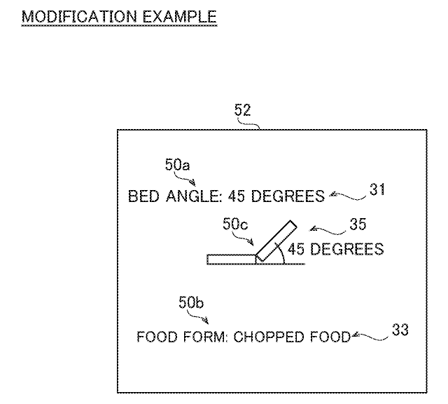

Next, with reference to FIG. 15, a process in which the control unit 410 stores the contact information 32c of the caregiver or the patient will be described.

In step 140a, the control unit 410 acquires the contact information 32c of the caregiver or the patient and the patient identification information 30 from the information terminal 8.

Next, in step 140b, the control unit 410 stores the contact information 32c of the caregiver or the patient and the patient identification information 30 in the first storage unit 11a in association with each other. Thereafter, the process ends.

Other configurations of the third embodiment are the same as those of the first embodiment described above.

### Modifications

It should be understood that the embodiments disclosed this time are illustrative in all respects and not restrictive. The scope of the present invention is indicated by the claims rather than by the description of the embodiments described above, and all modifications (variations) within the meaning and scope equivalent to the claims are included.

For example, in the system for a dysphagia patient, the angle information may be transmitted to each of the bed, the display device, and the information terminal.

Further, when transmitting the angle information to the bed, the angle information does not have to be displayed on the display device or the information terminal.

Further, the display device does not have to be provided in the vicinity of the bed as long as it is provided so as to be visible from the bed. For example, one display device may be provided for a patient room where a plurality of beds are arranged, and the configuration may be such that the angle information corresponding to each bed is transmitted to the display device.

Further, as long as the angle information is transmitted, the food form information does not have to be transmitted.

Further, the configuration may be such that the angle information is transmitted when it is time for the patient's meal, without depending on an input from the input receiving unit.

Further, the location information and the angle information may be stored in association with the patient identification information in one storage unit.

Further, the angle information may be transmitted to a bed (angle adjustment mechanism) or a display device provided in a facility different from the facility where the storage unit is provided.

Further, the control unit may transmit the angle information to an information terminal held by a caregiver who is in the same facility as the facility where the storage unit is provided.

Further, as long as the configuration is such that the angle information is transmitted, the configuration does not have to be such that the angle information is updated.

Further, when notifying that the bed angle is incorrect, the notification may be made by voice, light emission, or the like, or a combination of a message display and voice or the like may be used.

Further, notification that the bed angle is correct may be made.

Further, notification that the bed angle is incorrect does not have to be made.

Further, the configuration may be such that the contact information of the caregiver or the patient is acquired at a reception machine provided in a facility such as a hospital. In this case, the configuration may be such that the contact information of the caregiver or the patient is acquired when a QR code (registered trademark) of the reception machine is read by the information terminal.

Further, the contact information of the caregiver or the patient may be acquired during the patient's medical interview.

Further, the storage unit does not have to be provided in a facility such as a hospital.

Further, the control unit may be an information processing device provided separately from the hospital information system and the radiology information system.

Further, the patient identification information may be a My Number. In that case, the My Number stored in a storage unit other than the hospital information system and the radiology information system may be acquired and used as the patient identification information.

Further, when displaying the angle information on the display device or the information terminal, for example, as in a screen 52 shown in FIG. 16, a display field 50c for an illustration 35 showing the angle of the bed 6 may be provided together with the display field 50a and the display field 50b, and the illustration 35 showing the angle of the bed 6 may be displayed together with the angle information 31 and the food form information 33.

Further, although each process performed by the control unit has been described using a flow-driven flowchart that performs processing sequentially along the processing flow, the present invention is not limited to this. In the present invention, each process performed by the control unit may be performed by event-driven (event-driven type) processing that executes processing on an event basis. In this case, it may be performed in a completely event-driven manner, or a combination of event-driven and flow-driven may be used.

### Aspects

Those skilled in the art will understand that the exemplary embodiments described above are specific examples of the following aspects.

### Item 1

A system for a dysphagia patient, comprising: an information acquisition unit that acquires patient identification information capable of identifying the patient from an X-ray imaging apparatus and bed angle information of the patient's bed set based on the patient's swallowing function; a storage unit in which the patient identification information, location information indicating one of an address of the bed, an address of a display device installed in a location visible from the bed, and the patient's whereabouts, and the angle information are stored in association with each other; and a control unit, wherein the control unit: uses the patient identification information to acquire the location information and the angle information associated with the patient identification information from the storage unit; and uses the acquired location information to transmit the angle information to at least one of the bed, the display device, and an information terminal held by a caregiver for the patient's meal.

The bed angle information of the patient set based on the patient's swallowing function is transmitted to at least one of the patient's bed, the display device installed in a location visible from the bed, and the information terminal held by the caregiver for the patient's meal, using the location information associated with the patient identification information. This makes it possible to transmit the bed angle information from a doctor or a speech-language pathologist to a nurse or the like without involving a person using a memo or the like. As a result, it is possible to suppress the occurrence of human error related to the transmission of bed angle information when adjusting the bed angle of a patient with an impaired swallowing function.

### Item 2

The system for a dysphagia patient according to item 1, wherein the location information includes position information of the bed, position information of the display device, or contact information of the caregiver or the patient, and the control unit: in a case of transmitting the angle information to the bed, acquires the position information of the bed and the angle information associated with the patient identification information, and uses the acquired position information of the bed to transmit the angle information to the bed; in a case of transmitting the angle information to the display device, acquires the position information of the display device and the angle information associated with the patient identification information, and uses the acquired position information of the display device to transmit the angle information to the display device; and in a case of transmitting the angle information to the information terminal, acquires the contact information and the angle information associated with the patient identification information, and uses the acquired contact information to transmit the angle information to the information terminal.

When transmitting the angle information to the bed, by acquiring the position information of the bed, the angle information can be easily and reliably transmitted to the patient's bed. Further, when transmitting the angle information to the display device installed in a location visible from the patient's bed, by acquiring the position information of the display device, the angle information can be easily and reliably transmitted to the display device. Further, when transmitting the angle information to the information terminal, by acquiring the contact information of the caregiver or the patient, the angle information can be easily and reliably transmitted to the caregiver's information terminal. As a result, it is possible to further suppress the occurrence of human error related to the transmission of bed angle information when adjusting the patient's bed angle.

### Item 3

The system for a dysphagia patient according to item 2, wherein the control unit is configured to transmit the angle information to the display device or the information terminal, and the display device or the information terminal is configured to display the transmitted angle information.

Since the angle information is displayed on the display device, the caregiver can adjust the angle of the patient's bed while checking the angle information. As a result, it is possible to more effectively suppress the occurrence of human error related to the transmission of bed angle information when adjusting the patient's bed angle.

### Item 4

The system for a dysphagia patient according to item 3, further comprising an angle meter that is provided in a vicinity of the bed and acquires an angle of the bed, wherein the control unit is configured to, when the angle of the bed acquired by the angle meter is different from the angle information, notify that the angle of the bed is set to an incorrect angle.

When the angle of the bed is different from the angle information, that fact is notified, so that the caregiver can easily grasp that the angle of the bed is incorrect. As a result, when the angle of the bed is incorrect, the caregiver can readjust the angle of the bed, so that it is possible to further suppress the patient from having a meal with the angle of the bed being incorrect.

### Item 5

The system for a dysphagia patient according to item 3 or 4, wherein in the storage unit, food form information corresponding to the patient's swallowing function is stored in association with the patient identification information, and the control unit: uses the patient identification information to acquire the food form information associated with the patient identification information from the storage unit, together with the location information and the angle information; and transmits the acquired food form information, together with the angle information, to at least one of the display device and the information terminal.

Here, the food form of the patient's meal and the angle of the bed when having a meal are determined according to the patient's swallowing function. Therefore, if either the food form or the bed angle is not suitable for the patient's swallowing function, it is necessary to avoid letting the patient have a meal. Therefore, with the above configuration, the food form information is transmitted together with the angle information to at least one of the display device and the information terminal, so that the caregiver can check the food form information together with the angle information when adjusting the angle of the patient's bed. Therefore, if the meal served to the patient does not match the food form information, the caregiver can be notified in advance before the patient has a meal. As a result, it is possible to further suppress the patient from eating a meal in a form that is not suitable for the patient's swallowing function.

### Item 6

The system for a dysphagia patient according to any one of items 1 to 5, further comprising an input receiving unit that receives an operation input from an operator, wherein the control unit: uses the patient identification information input via the input receiving unit to acquire the location information and the angle information associated with the patient identification information from the storage unit; and uses the acquired location information to transmit the angle information to at least one of the bed, the display device, and the information terminal.

For example, when the caregiver performs an operation input to start adjusting the angle of the bed via the input receiving unit, the angle information is transmitted to at least one of the bed, the display device, and the information terminal. Therefore, when the caregiver assists the patient with a meal, the angle information can be transmitted to at least one of the bed, the display device, and the information terminal at the caregiver's desired timing. As a result, since the angle information can be transmitted at the timing desired by the caregiver, the caregiver's convenience (usability) can be improved.

### Item 7

The system for a dysphagia patient according to any one of items 1 to 6, wherein the storage unit includes a first storage unit included in a hospital information system and a second storage unit included in a radiology information system, in the first storage unit, the patient identification information and the location information are stored in association with each other, in the second storage unit, the patient identification information and the angle information are stored in association with each other, and the control unit: uses the patient identification information to acquire the location information associated with the patient identification information from the first storage unit; uses the patient identification information to acquire the angle information associated with the patient identification information from the second storage unit; and uses the acquired location information to transmit the angle information to at least one of the bed, the display device, and the information terminal.

Since the location information is stored in the first storage unit in association with the patient identification information, and the angle information is stored in the second storage unit in association with the patient identification information, even when the angle information and the location information are stored in different storage units, the corresponding angle information and location information can be easily acquired using the patient identification information.

### Item 8

The system for a dysphagia patient according to any one of items 2 to 7, further comprising an angle adjustment mechanism that adjusts an angle of the bed, wherein the control unit, in a case of transmitting the angle information to the bed, is configured to transmit the angle information to the angle adjustment mechanism, and the angle adjustment mechanism uses the transmitted angle information to adjust the angle of the bed.

Since the angle adjustment mechanism adjusts the angle of the bed using the angle information, the angle of the bed can be adjusted to an angle suitable for the swallowing function of the dysphagia patient without the caregiver adjusting the angle of the bed. As a result, it is possible to effectively suppress the occurrence of human error related to the transmission of bed angle information when adjusting the bed angle of a patient with an impaired swallowing function.

### Item 9

The system for a dysphagia patient according to any one of items 1 to 8, wherein the bed or the display device is provided in a same facility as a facility where the storage unit is provided, and the control unit is configured to transmit the angle information to the bed or the display device provided in the same facility as the facility where the storage unit is provided.

When the storage unit and the bed or display device are provided in the same facility such as a hospital, the angle information is transmitted to the bed or display device, so that it is possible to provide a system for a dysphagia patient that can suppress the occurrence of human error related to the transmission of bed angle information when adjusting the bed angle of a patient with an impaired swallowing function in a facility such as a hospital.

### Item 10

The system for a dysphagia patient according to any one of items 1 to 9, wherein the bed or the display device is provided in a different facility from a facility where the storage unit is provided, and the control unit is configured to transmit the angle information to at least one of the bed, the display device, and the information terminal provided in the different facility from the facility where the storage unit is provided.

Even when the storage unit is provided in a facility such as a hospital, and the bed or display device is provided in a facility different from the hospital, such as the patient's home, a nursing home, or a long-term care facility, the angle information is transmitted to the patient's home or the like, so that it is possible to provide a system for a dysphagia patient that can suppress the occurrence of human error related to the transmission of bed angle information when adjusting the bed angle of a patient with an impaired swallowing function in a facility such as a home.

### Item 11

The system for a dysphagia patient according to any one of items 1 to 10, wherein the control unit is configured to, when an examination by the X-ray imaging apparatus is repeated, acquire the angle information stored in the storage unit, and if there is a change in the angle information as a result of an examination of the patient's swallowing function, update the angle information.

When a change occurs in the patient's swallowing function, the angle information is updated to angle information corresponding to the change in the swallowing function, so that angle information for adjusting the bed to an angle suitable for the patient's swallowing function can be presented to the caregiver. As a result, even when the patient's swallowing function changes, it is possible to provide a system for a dysphagia patient that can suppress the occurrence of human error related to the transmission of bed angle information when adjusting the bed angle of a patient with an impaired swallowing function.

### Item 12

A method for a dysphagia patient, comprising the steps of: acquiring patient identification information capable of identifying the patient from an X-ray imaging apparatus and bed angle information of the patient's bed set based on the patient's swallowing function; storing the patient identification information, location information indicating one of an address of the bed, an address of a display device installed in a location visible from the bed, and the patient's whereabouts, and the angle information in association with each other; using the patient identification information to acquire the location information and the angle information associated with the patient identification information; and using the acquired location information to transmit the angle information to at least one of the bed, the display device, and an information terminal held by a caregiver for the patient's meal.

Similar to the system for a dysphagia patient described above, it is possible to provide a method for a dysphagia patient that can suppress the occurrence of human error related to the transmission of bed angle information when adjusting the bed angle of a patient with an impaired swallowing function.

### Item 13

A program for a dysphagia patient that causes a computer to execute the controls of: acquiring patient identification information capable of identifying the patient from an X-ray imaging apparatus and bed angle information of the patient's bed set based on the patient's swallowing function; storing the patient identification information, location information indicating one of an address of the bed, an address of a display device installed in a location visible from the bed, and the patient's whereabouts, and the angle information in association with each other; using the patient identification information to acquire the location information and the angle information associated with the patient identification information; and using the acquired location information to transmit the angle information to at least one of the bed, the display device, and an information terminal held by a caregiver for the patient's meal.

Similar to the system for a dysphagia patient described above, it is possible to provide a program for a dysphagia patient that can suppress the occurrence of human error related to the transmission of bed angle information when adjusting the bed angle of a patient with an impaired swallowing function.

### Reference Signs List

1, 301, 401 Hospital information system

2 Radiology information system

3 Display device

4 Input receiving unit

5 Angle meter

6 Bed

7 Angle adjustment mechanism

8 Information terminal

10 Information acquisition unit

11 Storage unit

11a First storage unit

11b Second storage unit

12, 310, 410 Control unit

30 Patient identification information

31 Angle information (patient's bed angle information)

32 Location information

32a Display device position information

32b Bed position information

32c Caregiver or patient contact information

40, 41, 42 Program (program for dysphagia patient)

80 Facility (facility where storage unit is provided)

81 Facility (facility different from facility where storage unit is provided)

100, 300, 400 System for dysphagia patient

200 X-ray imaging apparatus

1. A system for a dysphagia patient, comprising:
an information acquisition unit that acquires patient identification information capable of identifying a patient from an X-ray imaging apparatus and bed angle information of the patient's bed set based on the patient's swallowing function;
a storage unit in which the patient identification information, location information indicating one of an address of the bed, an address of a display device installed in a location visible from the bed, and the patient's whereabouts, and the angle information are stored in association with each other; and
a control unit, wherein
the control unit:
uses the patient identification information to acquire the location information and the angle information associated with the patient identification information from the storage unit; and
uses the acquired location information to transmit the angle information to at least one of the bed, the display device, and an information terminal held by a caregiver for the patient's meal.

2. The system for a dysphagia patient according to claim 1, wherein
the location information includes position information of the bed, position information of the display device, or contact information of the caregiver or the patient, and
the control unit:
in a case of transmitting the angle information to the bed, acquires the position information of the bed and the angle information associated with the patient identification information, and uses the acquired position information of the bed to transmit the angle information to the bed;
in a case of transmitting the angle information to the display device, acquires the position information of the display device and the angle information associated with the patient identification information, and uses the acquired position information of the display device to transmit the angle information to the display device; and
in a case of transmitting the angle information to the information terminal, acquires the contact information and the angle information associated with the patient identification information, and uses the acquired contact information to transmit the angle information to the information terminal.

3. The system for a dysphagia patient according to claim 2, wherein
the control unit is configured to transmit the angle information to the display device or the information terminal, and
the display device or the information terminal is configured to display the transmitted angle information.

4. The system for a dysphagia patient according to claim 3, further comprising
an angle meter that is provided in a vicinity of the bed and acquires an angle of the bed, wherein
the control unit is configured to, when the angle of the bed acquired by the angle meter is different from the angle information, notify that the angle of the bed is set to an incorrect angle.

5. The system for a dysphagia patient according to claim 3, wherein
in the storage unit, food form information corresponding to the patient's swallowing function is stored in association with the patient identification information, and
the control unit:
uses the patient identification information to acquire the food form information associated with the patient identification information from the storage unit, together with the location information and the angle information; and
transmits the acquired food form information, together with the angle information, to at least one of the display device and the information terminal.

6. The system for a dysphagia patient according to claim 1, further comprising
an input receiving unit that receives an operation input from an operator, wherein
the control unit:
uses the patient identification information input via the input receiving unit to acquire the location information and the angle information associated with the patient identification information from the storage unit; and
uses the acquired location information to transmit the angle information to at least one of the bed, the display device, and the information terminal.

7. The system for a dysphagia patient according to claim 1, wherein
the storage unit includes a first storage unit included in a hospital information system and a second storage unit included in a radiology information system,
in the first storage unit, the patient identification information and the location information are stored in association with each other,
in the second storage unit, the patient identification information and the angle information are stored in association with each other, and
the control unit:
uses the patient identification information to acquire the location information associated with the patient identification information from the first storage unit;
uses the patient identification information to acquire the angle information associated with the patient identification information from the second storage unit; and
uses the acquired location information to transmit the angle information to at least one of the bed, the display device, and the information terminal.

8. The system for a dysphagia patient according to claim 2, further comprising
an angle adjustment mechanism that adjusts an angle of the bed, wherein
the control unit, in a case of transmitting the angle information to the bed, is configured to transmit the angle information to the angle adjustment mechanism, and
the angle adjustment mechanism uses the transmitted angle information to adjust the angle of the bed.

9. The system for a dysphagia patient according to claim 1, wherein
the bed or the display device is provided in a same facility as a facility where the storage unit is provided, and
the control unit is configured to transmit the angle information to the bed or the display device provided in the same facility as the facility where the storage unit is provided.

10. The system for a dysphagia patient according to claim 1, wherein
the bed or the display device is provided in a different facility from a facility where the storage unit is provided, and
the control unit is configured to transmit the angle information to at least one of the bed, the display device, and the information terminal provided in the different facility from the facility where the storage unit is provided.

11. The system for a dysphagia patient according to claim 1, wherein
the control unit is configured to, when an examination by the X-ray imaging apparatus is repeated, acquire the angle information stored in the storage unit, and if there is a change in the angle information as a result of an examination of the patient's swallowing function, update the angle information.

12. A method for a dysphagia patient, comprising the steps of:
acquiring patient identification information capable of identifying a patient from an X-ray imaging apparatus and bed angle information of the patient's bed set based on the patient's swallowing function;
storing the patient identification information, location information indicating one of an address of the bed, an address of a display device installed in a location visible from the bed, and the patient's whereabouts, and the angle information in association with each other;
using the patient identification information to acquire the location information and the angle information associated with the patient identification information; and
using the acquired location information to transmit the angle information to at least one of the bed, the display device, and an information terminal held by a caregiver for the patient's meal.

13. A program for a dysphagia patient that causes a computer to execute the controls of:
acquiring patient identification information capable of identifying a patient from an X-ray imaging apparatus and bed angle information of the patient's bed set based on the patient's swallowing function;
storing the patient identification information, location information indicating one of an address of the bed, an address of a display device installed in a location visible from the bed, and the patient's whereabouts, and the angle information in association with each other;
using the patient identification information to acquire the location information and the angle information associated with the patient identification information; and
using the acquired location information to transmit the angle information to at least one of the bed, the display device, and an information terminal held by a caregiver for the patient's meal.
